(19) United States
(12) Patent Application Publication
(10) Pub. No.: US 2006/0084978 A1
(43) Pub. Date: Apr. 20, 2006

(54) Spinal fixation system and method

(76) Inventor: Mourad Ben Mokhtar, Paris (FR)

Correspondence Address: Carl M. Napolitano, Ph.D.;Allen, Dyer, Doppelt, Milbrath & Gilchrist, P.A., Suite 1401, 255 South Orange Avenue, Orlando, FL, 32801, US

(21) Appl. No.: 10/955,318

(22) Filed: Sep. 30, 2004

(51) Int. Cl. A61F 2/30

(52) U.S. Cl. 606/61

(57) ABSTRACT

A pedicle screw includes a cradle carrying a stabilization rod and a side entry channel for placing the rod into the cradle. A protuberance extends into the channel for providing an initial stop for the stabilization rod when being directed into the channel and is dimensioned for providing an audible sound through a snap fit of the stabilization rod in response to a biasing against the protuberance during a passing of the rod into the channel. An articulating ahead includes an insert carried within a cavity for making frictional contact with the stabilization rod and with the shank such that biasing a set screw against the rod results in a biasing of the rod against the insert and thus with the shank. Increasing the biasing by the setscrew against the rod results in a locking of the head with the shaft and thus a fixation of the stabilization rod.

### FIELD OF INVENTION

The invention generally relates to medical implants for spinal support, and more particularly to a pedicle screw for allowing ease in placement of a stabilizing rod for fixation of vertebrae.

### BACKGROUND

Internal fixation of the spine is well known, and as described in U.S. Pat. No. 5,474,555, internal fixation of the spine is a preferred method of immobilization since a patient will be allowed greater freedom when compared to external immobilization methods and the possibility of infection is greatly reduced. Further, pedicle devices are a favored means of attachment to vertebrae. As indicated in U.S. Pat. No. 5,466,237, internal fixation systems for vertebral fusion are preferably designed for ease of implantation and removal. Implants are relatively small and typically difficult to manipulate. Difficulty in assembling is exacerbated by the generally hostile environment in which a surgical operation must take place. It is well accepted that a fixation system should provide for ease in assembly of fixation elements while providing an option of removal.

As illustrated with reference to the above '237 and '555 patents describing a variable locking stabilizer anchor and a spinal implant system, respectively, pedicle screws of varying designs and methods of implantation are well known in the art. US patent application publication U.S. 2003/0144664 discloses a pedicle screw having an inclined channel for holding a support rod. A threaded plug secures the rod in the channel formed such that its longitudinal axis is inclined relative to the longitudinal axis of the threaded bone screw.

### SUMMARY

A spine fixation device, in keeping with the teachings of the present invention, may comprise a threaded shaft, a shank, and a connecting head attached to the shank. The head may include a cradle portion extending along an axis generally perpendicular to and intersecting a longitudinal axis of the shaft for receiving a stabilization rod. A channel is formed within a sidewall of the head and extends to the cradle portion. The channel portion may be dimensioned for closely receiving the stabilization rod. A protuberance extends into the channel for providing an initial stop for the stabilization rod when being directed into the channel. The protuberance is dimensioned for providing an audible sound through a snap fit of the stabilization rod in response to a biasing against the protuberance during a passing of the rod into the channel. A threaded bore extends along the longitudinal axis of the shaft and into the channel for receiving a threaded screw for securing the stabilization rod in the cradle portion of the head.

Alternatively, the head may be rotatably connected to the shank for providing an articulation thereof. The head may include a cavity having a generally concave surface with the shank comprising a generally convex surface so as to be rotatably carried within the cavity for articulation of the head with the shaft. An insert is carried within the cavity, wherein a surface of the insert forms a contact surface of the cradle portion for making frictional contact with the stabilization rod when carried in the cradle portion. An opposing surface of the insert is positioned for frictional contact with the convex surface of the shank such that a biasing of the threaded screw against the stabilization rod results in a biasing of the stabilization rod with the insert and subsequently with the shank. As a result, an increasing in the biasing by the threaded screw against the stabilization rod being caring in the cradler portion results in a locking of the head with the shaft and thus a fixation of the stabilization rod.

A method aspect of the invention may include attaching the fixation device to a spine and partially inserting a side wall of a stabilization rod into an entrance portion of the channel for biasing against the protuberance, increasing the biasing for moving the stabilization rod past the protuberance in a snap fit manner, moving the stabilization rod into the cradle, and securing the threaded screw against the stabilization rod for affixing the rod thereto.

### BRIEF DESCRIPTION OF DRAWINGS

For a fuller understanding of the invention, reference is made to the following detailed description, taken in connection with the accompanying drawings illustrating various embodiments of the present invention, in which:

FIG. 1 is a left rear perspective view of a pedicle screw having a rigid head illustrated with a stabilization rod carried thereby in keeping with the teachings of the present invention;

FIG. 2 is a front view of the embodiment depicted in FIG. 1;

FIG. 3 is a partial cross sectional view taken through a longitudinal axis and lines 3-3 of FIG. 2;

FIG. 4 is a partial enlarged cross sectional view of FIG. 3 illustrating elements of the embodiment of FIG. 1 including a channel positioned for side entry of a stabilization rod into a cradle for a securing therein;

FIG. 5 is a diagrammatical view illustrating a relationship between a channel and cradle of FIG. 4;

FIG. 6 is a side view of an alternate embodiment of the pedicle screw of FIG. 1 in keeping with the teachings of the present invention;

FIG. 7 is a front view of the embodiment of FIG. 6;

FIG. 8 is a partial cross sectional view taken through longitudinal axes of the shaft and an articulating head;

FIG. 9 is a partial enlarged cross sectional view of FIG. 8;

FIGS. 10, 11, and 12 are perspective, top plan, and side views, respectively, of an insert useful with the articulating head of FIG. 9;

FIG. 13 is a diagrammatical cross sectional view illustrating frictional contact surfaces cooperating for locking the head, stabilization rod, and shaft through a screwing action of a set screw against the stabilization rod carried within a cradle of the head; and

FIGS. 14, 15, 16, and 17 are perspective, bottom, side, and cross sectional views, respectively, for a cover useful with the embodiment of FIG. 6.

### DETAILED DESCRIPTION OF EMBODIMENTS

The present invention will now be described more fully with reference to the accompanying drawings in which alternate embodiments of the invention are shown and described. It is to be understood that the invention may be embodied in many different forms and should not be construed as limited to the illustrated embodiments set forth herein. Rather, these embodiments are provided so that this disclosure may be thorough and complete, and will convey the scope of the invention to those skilled in the art.

With reference initially to FIGS. 1 and 2, one embodiment of a spine fixation device 10, in keeping with the teachings of the present invention, includes a threaded shaft 12, a shank 14, and a connecting head 16 attached to the shank. The head 16 includes a multifaceted peripheral portion 17 for receiving a tool operable for rotating the device 10 in a screwing movement.

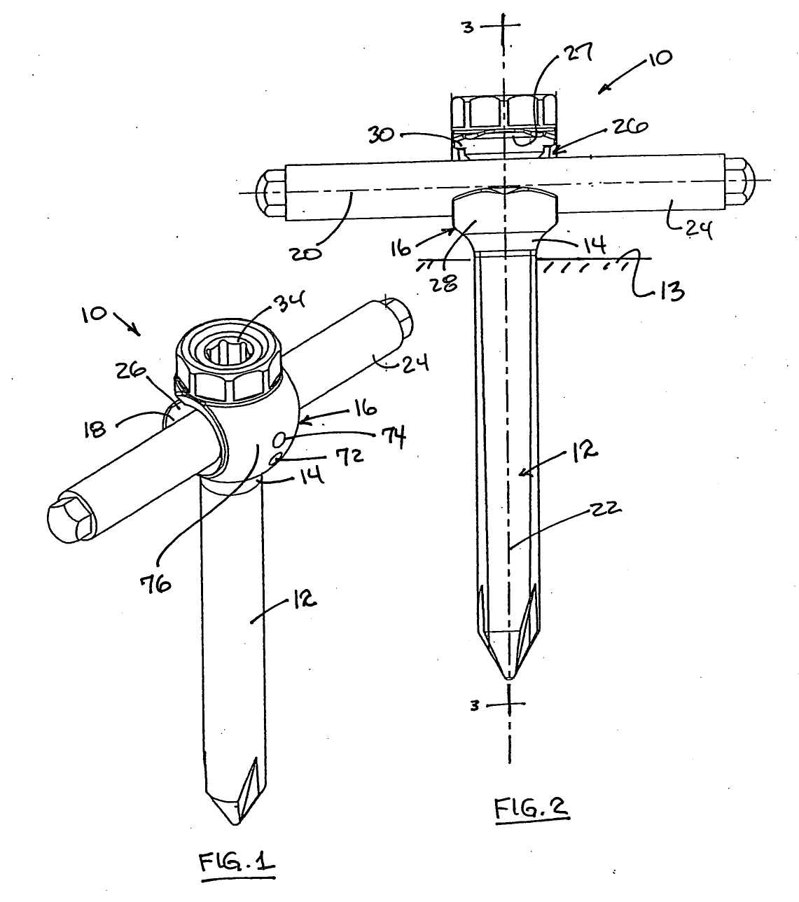

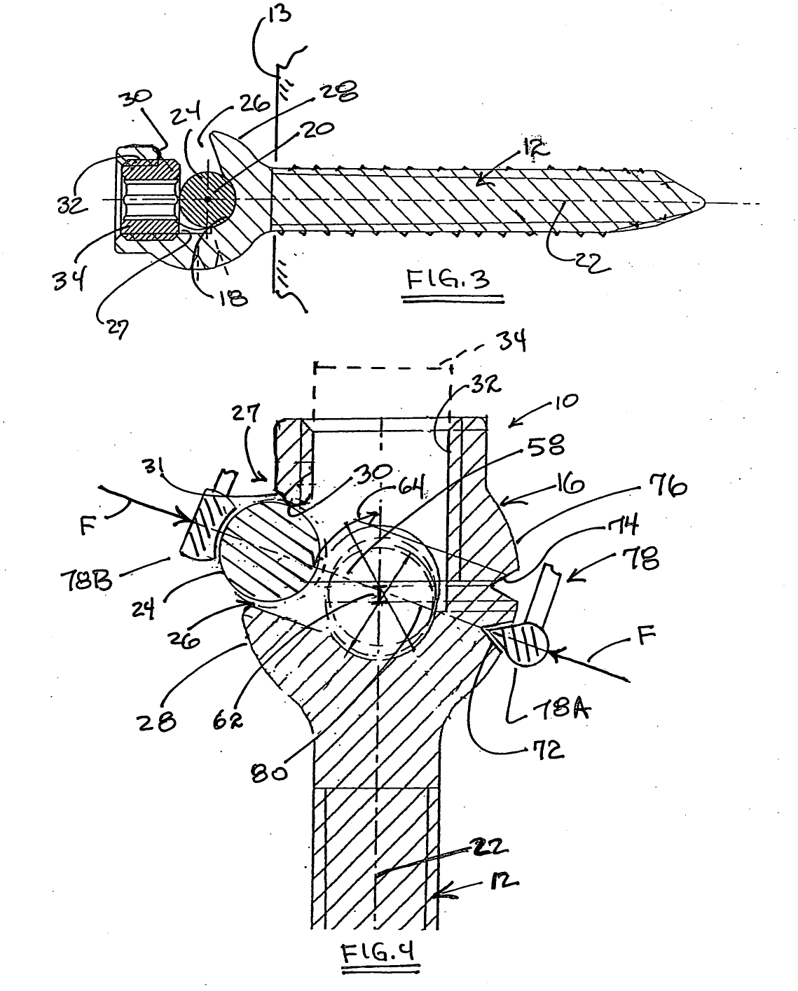

It is understood that the threaded shaft 12 may include various styled threads having a pitch as desired for typically screwing into the spine 13. The head 16 includes a cradle portion 18 extending along an axis 20 generally perpendicular to and intersecting a longitudinal axis 22 of the shaft 12 for receiving a stabilization rod 24, as illustrated with continued reference to FIGS. 1 and 2, and to FIG. 3. A channel 26 is formed within a sidewall 28 of the head 16 and extends to the cradle portion 18, as illustrated with reference again to FIG. 3 and to FIG. 4. The enlarged drawings of FIG. 4 dimensioned by way of example. For the embodiment herein described by way of example, the channel portion 18 is dimensioned for closely receiving the stabilization rod 24.

A protuberance 30 extends into the channel 26 for providing an initial stop for the stabilization rod 24 when being directed into the channel 26. The protuberance 30 is dimensioned for providing an audible sound through a snap fit of the stabilization rod 24 in response to a biasing against the protuberance during a passing of the rod (24a to 24b) into the channel 26, as illustrated with reference to FIG. 5. With reference again to FIGS. 3 and 4, a threaded bore 32 extends along the longitudinal axis 22 of the shaft 12 and opens into the an upper portion 27 of the channel 26 for receiving a threaded screw such as a set screw 34 that secures the stabilization rod 24 in the cradle portion 18 of the head 16. The setscrew 34 has a diameter substantially greater than a diameter of the stabilization rod 24.

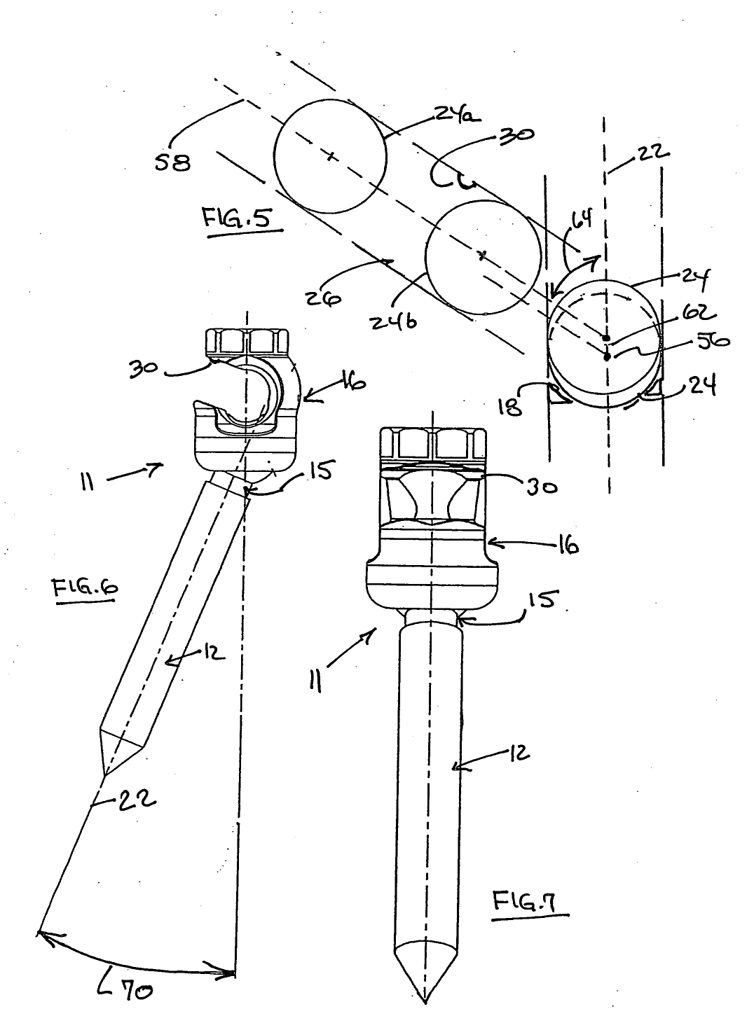

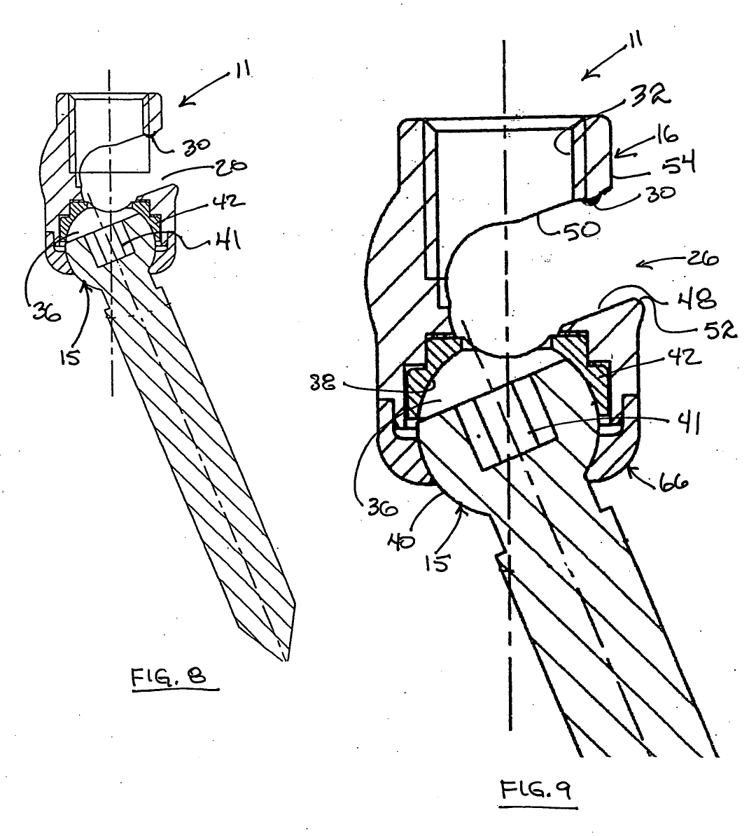

For a second embodiment of a spine fixation device 11, and with reference to FIGS. 6 and 7, the head 16 may be rotatably connected to the alternate shank 15 for providing an articulation thereof. As herein described by way of example, the head 16 includes a cavity 36 having a generally concave surface 38. The shank 15 comprises a generally convex surface 40 so as to be rotatably carried within the cavity 36 for articulation of the head 16 with the shaft 12. As illustrated with reference to FIGS. 8 and 9, the cavity 36 and shank 15 cooperate to allow articulation using generally spherical shapes for the surfaces 38, 40. The shank 15 includes a bore 41 therein having a multifaceted wall for receiving a tool for screwing the threaded shaft 12 into a bone, by way of example.

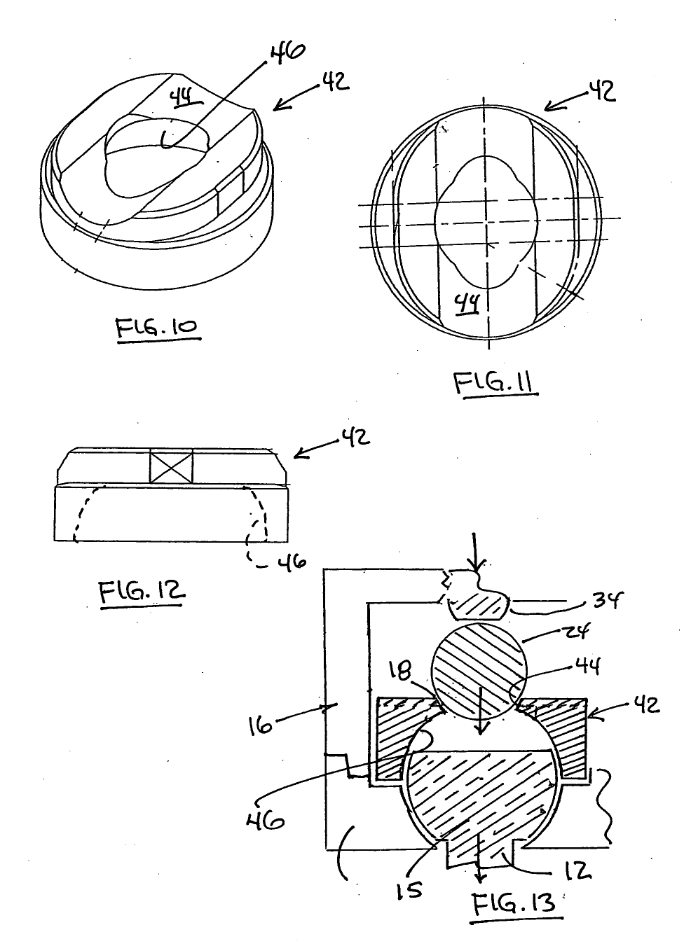

With continued reference to FIGS. 8 and 9, and to FIGS. 10, 11, and 12, an insert 42 is carried within the cavity 36, wherein a surface portion 44 of the insert forms a contact surface of the cradle portion 18 for making frictional contact with the stabilization rod 24 when the rod is in the cradle portion. An opposing surface 46 of the insert 42 is positioned for frictional contact with the convex surface 40 of the shank 15 such that a biasing of the set screw 34 against the stabilization rod 24 results in a biasing of the stabilization rod with the insert and thus with the shank. As a result, and as illustrated with reference to FIG. 13, an increasing in the biasing by the threaded set screw 34 against the stabilization rod 24 being caring in the cradler portion 18 results in a locking of the head 16 with the shaft 12 and thus a fixation of the stabilization rod.

By way of further discussion, and with reference again to FIG. 9, by way of example, the channel 26 is formed by a lower surface 48 being proximate the shank 15 and an opposing upper surface 50 proximate the threaded bore 32. The lower surface 48 extends outward from the longitudinal axis 22 beyond the upper surface 50 for providing a shelf edge 52 for guiding the stabilization rod 24 into the channel 26, as further illustrated with reference again to FIG. 4. For the embodiment herein described, the protuberance 30 extends from the upper surface 50 of the channel 26. Further, the protuberance 30 is carried in a spaced relation 31 to an outside wall portion 54 of the head 16.

With reference again to FIGS. 4 and 5, and by way of example, the cradle 18 for the embodiment herein described comprises a circular arc for receiving the stabilization rod 24, wherein a center 56 of a circle defining the arc is within the longitudinal axis 22. A central axis 58 of the channel 26 is coincident with a longitudinal axis of the stabilization rod 24 during travel therethrough, and an intersection 60 of the central axis 58 of the channel 26 and the longitudinal axis 22 of the shaft 12 is offset 62 from the center 56 of the circle defining the arc of the cradle portion 18. In one embodiment, the central axis 58 of the channel 26 forms an angle 64 between sixty and eighty degrees with the longitudinal axis 22.

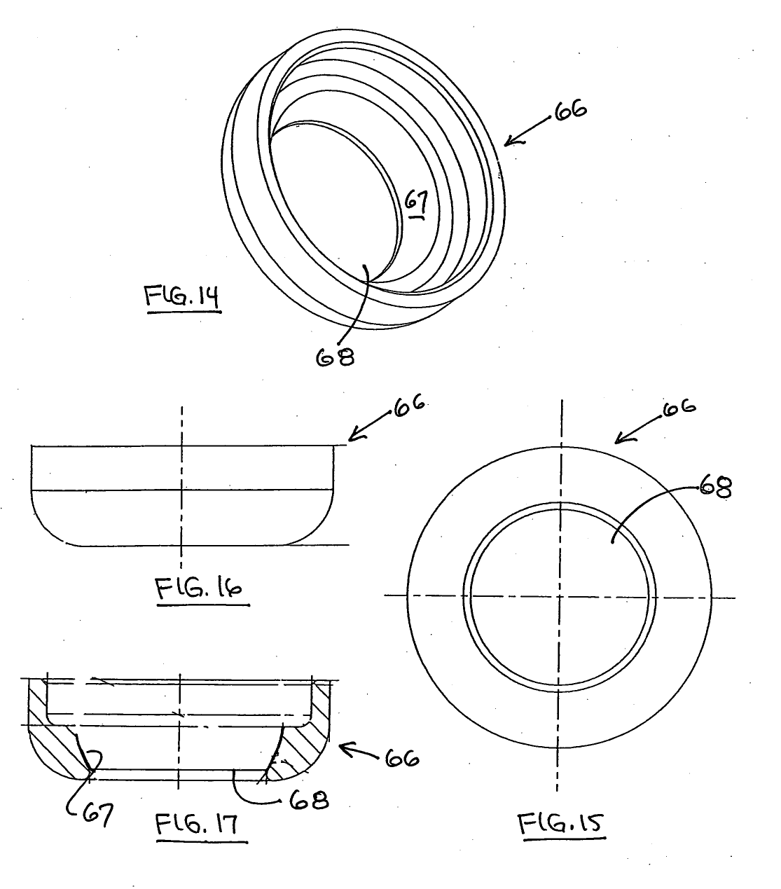

With reference again to FIGS. 6-9, and with reference to FIGS. 14-17, the head 16 comprising a cover 66 having an aperture 68 therein for receiving the shaft 12 therethrough. The cover 66 is fitted as part of the head 16 for confining the shank 15 to within the cavity 36. An inside surface portion 67 of the cover 66 comprises a spherical shape for cooperating with the convex surface of the shank 15 earlier described with reference to FIG. 9. The aperture 68 is sized for limiting rotation of the head to within an angle 70 of approximately twenty-five degrees from the longitudinal axis 22.

With reference again to FIGS. 1 and 4, by way of example, one embodiment of the fixation device 10 may include indentations 72, 74 within a sidewall portion 76 opposing the channel 26. The indentations provide a well-defined point of contact for a jaw styled tool 78 wherein opposing jaw elements 78A, 78B are biased F against at least one indentation 72 and against the stabilization rod 24 during an inserting of the rod into the channel 26 and past the protuberance 30. As illustrated with continued reference to FIG. 4, a line 80 extending through the central axis 58 of the channel extends through the indentation 72.

Many modifications and other embodiments of the invention will come to the mind of one skilled in the art having the benefit of the teachings presented in the foregoing descriptions and the associated drawings. Therefore, it is to be understood that the invention is not to be limited to the specific embodiments disclosed, and that modifications and alternate embodiments are intended to be included within the scope of the appended claims.

That which is claimed is:

1. A spine fixation device comprising:
a shaft having a threaded screw portion at a distal end for fixing to a bone, and an opposing shank at a proximal end thereof;
a head attached to the shank, the head comprising:
a cradle portion extending therethrough along an axis generally perpendicular to and intersecting with a longitudinal axis of the shaft for receiving a stabilization rod therein;
a channel formed within a side wall thereof and extending into the head to the cradle portion, wherein the channel is dimensioned for closely receiving the stabilization rod for placement into the cradle portion, and wherein a protuberance extends into the channel for providing an initial stop for the stabilization rod being directed into the channel, the protuberance dimensioned for providing a snap fit of the stabilization rod in response to a biasing thereagainst when passing into the channel; and
a threaded bore therein extending along the longitudinal axis of the shaft into the channel; and
a threaded screw operable within the threaded bore for securing the stabilization rod in the cradle portion of the head.

2. A fixation device according to claim 1, wherein the channel is formed by a lower surface thereof proximate the shank and an opposing upper surface proximate the threaded bore, and wherein the lower surface extends outward from the longitudinal axis of the shaft beyond the upper surface for providing a shelf portion for guiding the stabilization rod into the channel.

3. A fixation device according to claim 2, wherein the protuberance extends from the upper surface of the channel.

4. A fixation device according to claim 3, wherein the protuberance is carried in a spaced relation to an outside wall of the head.

5. A fixation device according to claim 1, wherein the cradle comprises a circular arc for receiving the stabilization rod, and wherein a center of a circle defining the arc is within the longitudinal axis of the shaft.

6. A fixation device according to claim 5, wherein a central axis of the channel is coincident with a longitudinal axis of the stabilization rod during travel therethrough, and wherein an intersection of the central axis of the channel and the longitudinal axis of the shaft is offset from the center of the circle defining the arc of the cradle.

7. A fixation device according to claim 6, wherein the central axis of the channel forms an angle between sixty and eighty degrees with the longitudinal axis as measured from the top portion of the head.

8. A fixation device according to claim 1, wherein the threaded screw comprises a diameter substantially greater than a diameter of the stabilization rod.

9. A fixation device according to claim 1, wherein the head further comprises a multifaceted peripheral portion for receiving a tool operable for rotating the device in a screwing movement therefor.

10. A fixation device according to claim 1, wherein the head is rotatably connected to the shank.

11. A fixation device according to claim 10, wherein the head comprises a cavity having a generally concave surface, and wherein the shank comprises a generally convex surface rotatably carried within the cavity for articulation of the head with the shaft.

12. A fixation device according to claim 11, further comprising an insert carried within the cavity, wherein a surface of the insert forms a contact surface of the cradle portion for making frictional contact with the stabilization rod carried therein, and wherein an opposing surface of the insert is positioned for frictional contact with the convex surface of the shank such that a biasing of the threaded screw against the stabilization rod results in a biasing of the stabilization rod with the insert and subsequently with the shank, an increasing in the biasing resulting in a locking of the head with the shaft and thus a fixation of the stabilization rod thereby.

13. A fixation device according to claim 11, further comprising a cover having an aperture therein for receiving the shaft therethrough, the cover fitted with the head for confining the shank to the cavity.

14. A fixation device according to claim 13, wherein the aperture is sized for limiting rotation of the head to with approximately twenty five degrees from the longitudinal axis of the shaft.

15. A fixation device according to claim 10, wherein the shank includes a bore therein having a multifaceted wall for receiving a tool for screwing the shaft having the threaded screw portion into a bone.

16. A fixation device according to claim 1, wherein the head further comprises an indentation within a sidewall portion opposing the channel, the indentation operable for receiving a stabilization rod insertion tool wherein one portion of the tool is biased against the indentation and an opposing portion is biased against the stabilization rod during an inserting of the rod into the channel and past the protuberance.

17. A fixation device according to claim 16, wherein a line extending through a central axis of the channel extends through the indentation.

18. A spine fixation device comprising:
a head having a channel extending into the head through a sidewall portion thereof, wherein the channel is dimensioned for closely receiving a stabilization rod, and wherein a protuberance extends into the channel for providing an initial stop for the stabilization rod being directed into the channel, the protuberance dimensioned for providing a snap fit of the stabilization rod in response to a biasing thereagainst when passing into the channel, the head further having a threaded bore extending along a longitudinal axis thereof; and
a threaded screw operable within the threaded bore for securing the stabilization rod to the head.

19. A fixation device according to claim 18, further comprising a cradle extending through a head portion along an axis generally perpendicular to and intersecting with the longitudinal axis.

20. A fixation device according to claim 18, wherein the channel is formed by a lower surface thereof and an opposing upper surface, and wherein the lower surface extends outward from the longitudinal axis beyond the upper surface for providing a shelf portion for guiding the stabilization rod into the channel.

21. A fixation device according to claim 20, wherein the protuberance extends from the upper surface of the channel.

22. A fixation device according to claim 21, wherein the protuberance is carried in a spaced relation to an outside wall of the head.

23. A fixation device according to claim 18, wherein a central axis of the channel forms an angle between sixty and eighty degrees with the longitudinal axis.

24. A fixation device according to claim 18, wherein the threaded screw comprises a diameter substantially greater than a diameter of the stabilization rod.

25. A fixation device according to claim 18, wherein the head further comprises a multifaceted peripheral portion for receiving a tool operable for rotating the device in a screwing movement therefor.

26. A fixation device according to claim 18, wherein the head is rotatably connected to a shank.

27. A fixation device according to claim 26, wherein the shank includes a bore therein having a multifaceted wall for receiving a tool for screwing the shaft having the threaded screw portion into a bone.

28. A fixation device according to claim 18, wherein the head further comprises an indentation within a sidewall portion opposing the channel, the indentation operable for receiving a stabilization rod insertion tool, wherein one portion of the tool is biased against the indentation and an opposing portion is biased against the stabilization rod during an inserting of the rod into the channel and past the protuberance.

29. A fixation device according to claim 28, wherein a line extending through a central axis of the channel extends through the indentation.

30. A method of inserting a stabilization rod into a fixation device having a side entry channel within a head portion thereof, wherein a wall of the channel includes a protuberance extending therefrom for providing a snap fit of the rod passing into the channel, the fixation device further having a cradle portion within the head and a threaded screw for securing the rod to the device, the method comprising:
attaching the fixation device to a spine;
providing a stabilization rod;
partially inserting a side wall of the stabilization rod into an entrance portion of the channel for biasing against the protuberance;
increasing the biasing for moving the stabilization rod past the protuberance in a snap fit manner;
moving the stabilization rod into the cradle; and
securing the threaded screw against the stabilization rod for affixing the rod thereto.

31. A method according to claim 30, wherein the head comprises an indentation within a sidewall portion opposing the channel, the method further comprising:
biasing one portion of a rod insertion tool against the indentation;
biasing an opposing portion of the insertion tool against the stabilization rod; and
forcing the rod into the channel past the protuberance through the biasing.
